(19) United States
(12) Patent Application Publication
(10) Pub. No.: US 2005/0288554 A1
(43) Pub. Date: Dec. 29, 2005

(54) Endoscope

(75) Inventors: Kunihiko Miyagi, Wako-shi (JP); Masahiro Inoue, Soka-shi (JP)

Correspondence Address: OSHA LIANG L.L.P., 1221 MCKINNEY STREET, SUITE 2800, HOUSTON, TX, 77010, US

(73) Assignee: Machida Endoscope Co., Ltd., Tokyo (JP)

(21) Appl. No.: 11/148,148

(22) Filed: Jun. 8, 2005

(30) Foreign Application Priority Data
Mar. 12, 2003 (JP) 2003-066055

(51) Int. Cl. A61B 1/00

(52) U.S. Cl. 600/129; 600/117; 600/182

(57) ABSTRACT

An endoscope 10 comprises an insert portion 30 having outer tubing 35, a light guide 31, an image guide 32 and an objective optical member 33. Peripheral side surfaces of these components 31,32,33,35 adjacent to each other are bonded together by an adhesive 36 at a distal end portion of the insert portion 30. The adhesive 36 contains fine particles 36b comprising a roentgenopaque material such as Pt or Au. The adhesive 36 fills among optical fibers 31a inside the light guide 31, thereby roentgenopaque property is conferred not only among the above-mentioned components 31,32,33,35 at the distal end of the insert portion 30 but also on the inside of the light guide 31. This enables the distal end of the endoscope 10 to be traced with X-rays without enhancing the diameter of the insert portion 30. Therefore, the endoscope 10 can easily be inserted into human small vessels such as blood vessels.

### FIELD OF THE INVENTION

The present invention relates to an endoscope suitable to be inserted into extremely small vessels like human small vessels such as blood vessels and lymphatic vessels, and more particularly relates to an endoscope whose distal end is traceable with X-rays.

### DESCRIPTION OF THE RELATED ART

Extremely fine endoscopes which can be inserted in blood vessels are known. When observing a coronary artery of the heart, for example, with such an endoscope, it is important to monitor the position of distal end of the endoscope. A band called x-ray marker provided around a distal end portion of an insert portion is a known solution to meet this requirement. The band is made of metal such as platinum, and thus can be detected with X-rays.

Patent references 1 and 2 disclose ordinary-sized endoscopes whose diameters are too big to be inserted into blood vessels, but the locations of whose distal ends can be detected with X-rays by virtue of metal powders contained in plastic tip covers at the distal ends thereof.

Patent Reference 1: Japanese Patent Application Laid-Open No. H8-243072

Patent Reference 2: Japanese Patent Application Laid-Open No. 2002-112951

When an X-ray marker band is disposed around an intravascular endoscope, the thickness of the band increases the diameter of the intravascular endoscope and makes it less easy for the endoscope to be inserted into blood vessels. In some intravascular endoscopes, an X-ray band is inlaid into outer tubing of an insert portion. In such an arrangement, structural stress is applied to the site where the band is inlaid, which reduces durability of the endoscope. A certain amount of size is required for the tip covers mentioned in the above references, so that the tip covers aren't suitable to be applied to an extremely fine endoscope such as the one for intravascular endoscopy. Furthermore, use of the band or the tip covers involves an increase in the number of components, which requires additional steps in the assembly process.

### BRIEF SUMMARY OF THE INVENTION

The present invention has been accomplished in order to solve the above-mentioned problems. According to the present invention, there is provided an endoscope whose distal end is traceable with X-rays comprising: an insert portion having a light guide, an image guide, an objective optical member disposed at a distal end of the image guide, and outer tubing encasing the light guide, the image guide and the objective optical member; and an adhesive that is disposed at an inside of a distal end region of the insert portion in such a manner as to fill around the light guide, the image guide and the objective optical member in the outer tubing, wherein the adhesive has roentgenopaque property.

Owing to the features described above, the distal end of the endoscope can be traced with X-rays without increasing the diameter of the insert portion. As a result, the endoscope can be easily inserted into even extremely small vessels such as human small vessels for observation.

Preferably, roentgenopaque fine particles are contained in the adhesive, thereby providing a roentgenpaque adhesive.

Preferably, the light guide is comprised of a bundle of a plurality of optical fibers, and the adhesive fills among the optical fibers at the distal end region of the insert portion, thereby roentgenopacity is given to the inside of the light guide.

### BRIEF DESCRIPTION OF THE DRAWINGS

FIG. 1 is a side view of an embodiment of an intravascular endoscope in accordance with the present invention.

FIG. 2 is a sectional side view of the distal end region of the insert portion of the intravascular endoscope illustrated in FIG. 1.

FIG. 3 is a front view along line III-III of FIG. 2 of the distal end surface of the insert portion.

FIG. 4 is a cross-sectional view along line IV-IV of FIG. 2 of the distal end region of the insert portion.

FIG. 5 is a perspective view showing a part of the distal end region of the insert portion.

### DETAILED DESCRIPTION OF THE INVENTION

One preferred embodiment of the present invention will be described hereinafter with reference to the accompanying drawings.

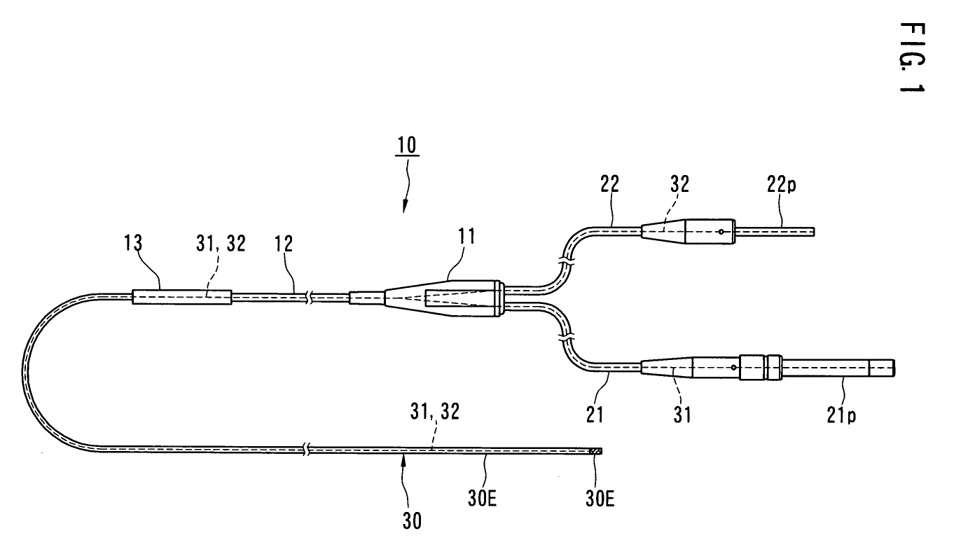

FIG. 1 illustrates an intravascular endoscope 10 suitable for observation of the inner walls of blood vessels (human small vessels) such as coronary arteries of the heart. The endoscope 10 comprises a grip 11, a relay sleeve 13 connected to a distal end of the grip 11 via an intermediate cable 12, a flexible insert portion 30 extending in length from the relay sleeve 13, and two cables 21, 22 extending from a basal end of the grip 11. A light plug 21b is attached to an end of the light guide cable 21. The light plug 21b is to be connected to a light source (not shown). An image plug 22p is attached to an end of the image guide cable 22. The image plug 22p is to be connected to a TV camera unit (not shown).

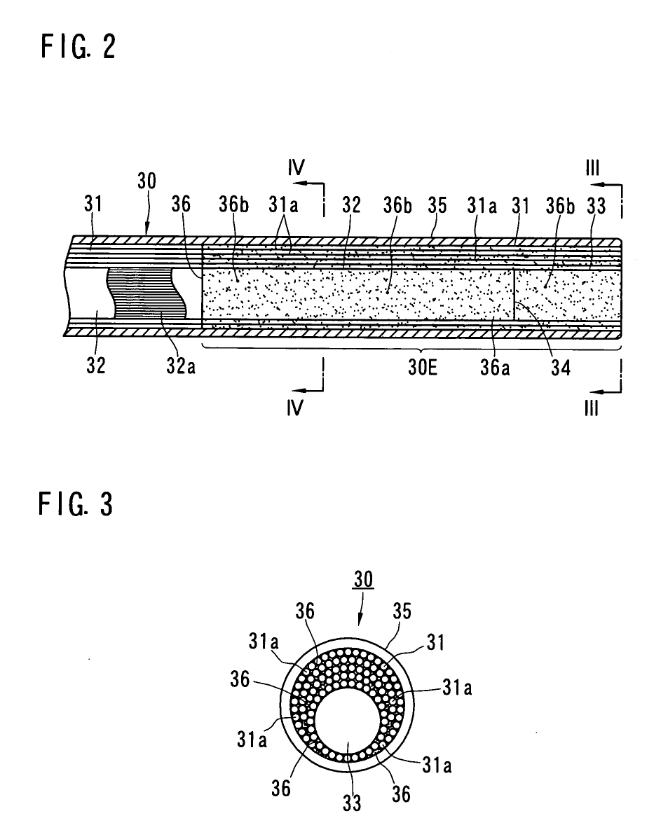

As shown in FIGS. 2 to 4, the insert portion 30 has outer tubing 35. A light guide 31, an image guide 32, and a Selfoc lens 33 as an objective optical member are encased in the outer tubing 35. The outer tubing 35, in other words, the insert portion 30, is long enough to reach from a blood vessel in a human thigh to a coronary artery of the heart, for example. Outer diameter of the outer tubing 35, in other words, outer diameter of the insert portion 30, is sufficiently smaller than inner diameter of an artery, being in the order of several tenths of a millimeter. Wall thickness of the outer tubing 35 is in the order of several tens of micrometers.

In general, when observing a coronary artery with an endoscope, a guiding wire is inserted from an artery in a thigh or an upper arm to a target site in the heart. Next, a guiding catheter is inserted over the guiding wire into the artery. Then, the guiding wire is removed while the guiding catheter is left in the artery. An insert portion of the endoscope is inserted into the guiding catheter staying in the artery. Therefore, the outer diameter of the insert portion 30 of the endoscope 10 is not only smaller than the inner diameter of the artery but also smaller than the inner diameter of the guiding catheter.

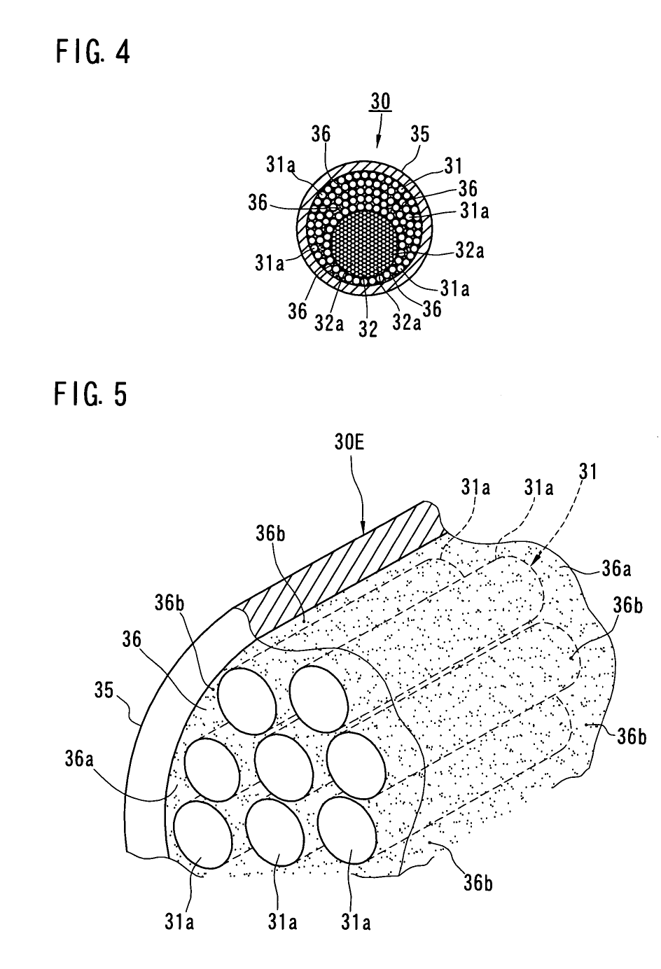

As shown in FIGS. 2 and 4, the image guide 32 of the endoscope 10 comprises a bundle of a plurality of optical fibers 32a. These optical fibers 32a are fusion-spliced to form a single conduit. The diameter of the image guide 32 is about a half of that of the outer tubing 35, for example. As shown in FIG. 1, the image guide 32 extends from inside the outer tubing 35 of the insert portion 30 through the relay sleeve 13, the relay cable 12, the grip 11, and the image cable 22. A basal end of the image guide 32 extends to the image plug 22p.

As shown in FIG. 2, a distal end of the image guide 32 recedes a little (about 1 mm, for example) from a distal end of the insert portion 30. The Selfoc lens 33 is bonded onto the distal end surface of the image guide 32 by a transparent adhesive 34 which is different from an adhesive 36 as described later. As shown in FIGS. 2 and 3, the Selfoc lens 33 has a columnar shape with generally the same diameter as the image guide 32. The Selfoc lens 33 is disposed at the distal end of the image guide 32 to form a continuous straight line from the image guide 32. A distal end surface of the Selfoc lens 33 is exposed to outside in the same plane as distal end surfaces of the outer tubing 35 and the light guide 31, serving as an observation port for image light incident from an object of observation. The Selfoc lens 33 focuses the incident light onto the distal end surface of the image guide 32.

As shown in FIGS. 2 and 4, the light guide 31 is comprised of a bundle of a plurality of optical fibers 31a. The optical fibers 31a of the light guide 31 do not form a conduit, different from the case with the optical fibers 32a of the image guide 32. As shown in FIGS. 3 and 4, the light guide 31 is arranged to surround the image guide 32 and the Selfoc lens 33 in the insert portion 30. The distal end surface of the light guide 31 is exposed to outside in the same plane as the distal end surfaces of the outer tubing 35 and the Selfoc lens 33. As shown in FIG. 1, the light guide 31 extends from inside the outer tubing 35 of the insert portion 30 through the relay sleeve 13, the relay cable 12, the grip 11, and the light cable 21. A basal end of the light guide 31 extends to the light plug 21p.

Illumination light from the light source mentioned above travels through the light guide 31, emerges out of the distal end surface of the light guide 31 and illuminates the object of observation such as the inside of a blood vessel. Image light from the illuminated object of observation is made incident to the Selfoc lens 33, and is transmitted through the image guide 32 to the TV camera unit. The optical to electrical signal conversion is performed on the image light in the TV camera unit, followed by the electrical to video signal conversion. Thereby, the image of the object of observation can be shown on a monitor screen.

As shown in FIG. 2, peripheral side surfaces of components 31, 32, 33 and 35 adjacent to each other are bonded together by the adhesive 36 at a distal end region 30E of the insert portion 30 (a region up to approximately 3 mm from the distal end surface, for instance). More specifically, as shown in FIGS. 3 and 4, the adhesive 36 is filled between an inner peripheral surface of the outer tubing 35 and the light guide 31, between the light guide 31 and the Selfoc lens 33 and between the light guide 31 and an outer peripheral surface of the image guide 32.

The adhesive 36 comprises a main adhesive agent 36a having an adhesive action and roentgenopaque fine particles 36b mixed in the main adhesive agent 36a. The main adhesive agent 36a is comprised of black epoxy resin, for example. Black-colored resin is suitable because it prevents illumination light traveling through light guide 31 from being made incident to the Selfoc lens 33, and consequently prevents degradation of contrast in image.

The fine particles 36b in the adhesive 36 are comprised of a roentgenopaque material such as a metal like platinum (Pt) or gold (Au). This confers roentgenopaque property on the adhesive 36, and consequently on the distal end region 30E of the insert portion 30.

Furthermore, as shown in FIG. 5, the adhesive 36 containing the fine particles 36b fills among optical fibers 31a forming the light guide, bonding the optical fibers 31a together. This confers roentgenopaque property on the inside of the light guide 31. Optical fibers 31a may occupy approximately 70% of cross-sectional area of the light guide 31 with remaining 30% being occupied by the adhesive 36, for example.

In the endoscope 10 thus constructed, roentgenopaque property is conferred on the distal end region 30E of the insert portion 30 by the adhesive 36 containing fine particles 36b. As a result, when performing an endoscopic observation of the inside of the coronary artery of the heart, for example, the position of the distal end of the insert portion 30 can be monitored from outside the body of the patient by roentgenoscopy.

The fine particles 36b can be distributed in large quantity and widely in three dimensions because the adhesive 36 fills inside the light guide 31 as well as among the peripheral side surfaces of components 31, 32, 33 and 35 adjacent to each other. Consequently, roentgenopaque property of the distal end region 30E of the insert portion 30 can be further enhanced. As a result, X-ray monitoring of the distal end position can be performed further reliably.

As apparent from the foregoing description, outer diameter of the distal end region 30E of the insert portion 30 can be as extremely small as that of the portions nearer to the basal end. This makes the insert portion 30 sufficiently easy to be inserted.

As mentioned above, the intravascular endoscope 10 for coronary arteries is inserted into the guiding catheter. Therefore, smallness in diameter in the distal end region 30E of the endoscope insert portion 30 makes it possible to make the diameter of the guiding catheter small enough to be easily inserted into a blood vessel.

Since portions including the outer tubing 35 of the insert portion 30 are free from local structural stress, durability of the endoscope can be enhanced.

The endoscope 10 uses the same components as ordinary extremely-small-diameter intravascular endoscopes without roentgenopaque property, and thus an increase in the number of components can be avoided. Conferring roentgenopaque property in this way does not involve substantial increase in complexity in manufacturing process. The endoscope 10 is manufactured in the normal process except that the fine particles 36b should be added to and mixed with the adhesive 36 beforehand.

It should be noted that the present invention is not limited to the above embodiment but that various applications and modifications may be made within the scope of the invention.

For example, the present invention is applicable not only to observation of coronary arteries but also to observation of other blood vessels in other parts of the human body. It is also applicable to observation of other human small vessels such as lymphatic vessels and pancreatic ducts. Moreover, its application is not limited to observation of human vessels, but extends to observation of structures in general with extremely small channels.

The objective optical member may comprise an object lens which is optically connected to the image guide and an observation port which is a separate component from the lens and exposed on the distal end surface of the insert portion.

The main adhesive agent 36a having an adhesive action may itself be roentgenopaque.

1. An endoscope whose distal end is traceable with X-rays comprising:
an insert portion having a light guide, an image guide, an objective optical member disposed at a distal end of said image guide, and outer tubing encasing said light guide, said image guide and said objective optical member; and
an adhesive that is disposed at an inside of a distal end region of said insert portion in such a manner as to fill around said light guide, said image guide and said objective optical member in said outer tubing, wherein said adhesive has roentgenopaque property.

2. An endoscope according to claim 1, wherein roentgenopaque fine particles are contained in said adhesive.

3. An endoscope according to claim 1, wherein said light guide is comprised of a bundle of a plurality of optical fibers, and said adhesive fills among said optical fibers at the distal end region of said insert portion, thereby roentgenopacity is given to the inside of said light guide.
